(19) United States
(12) Patent Application Publication
(10) Pub. No.: US 2021/0346123 A1
(43) Pub. Date: Nov. 11, 2021

(54) DENTAL LASER TREATMENT APPARATUS

(71) Applicant: Epios Co., Ltd., Tokyo (JP)

(72) Inventor: Yasuo Shichitani, Tokyo (JP)

(21) Appl. No.: 16/322,743

(22) PCT Filed: Aug. 1, 2016
(86) PCT No.: PCT/JP2016/003538

§ 371 Date: Feb. 1, 2019

(51) Int. Cl. A61C 1/00; A61C 17/00; A61L 2/00; A61L 2/26; B23K 26/14

(52) U.S. Cl. CPC A61C 1/0046; A61C 17/00; A61L 2/0029; A61L 2101/06; A61L 2/26; B23K 26/14; A61L 2/0088

(57) ABSTRACT

Provided is a dental laser treatment apparatus that can facilitate sterilization and cleaning work in a treatment with laser beams and also during and after the treatment, which puts less physical and mental burdens on a patient, offers a prospect of quick and reliable therapeutic effects, and allows for faster healing. A dental laser treatment apparatus may include an apparatus body including a laser beam generating device that generates a laser beam; a laser beam application device including an optical fiber that supplies the laser beam generated by the laser beam generating device and a handpiece that includes an application port for the laser beam attached on a distal portion of the optical fiber; and a cleaning solution supply unit that discharges sterilizing/cleaning solution from a sterilizing/cleaning solution supply unit.

### CROSS-REFERENCE TO RELATED APPLICATION

This application claims priority to International Application PCT/JP2016/003538 filed on Aug. 1, 2016, the contents of which are hereby incorporated by reference in its entirety.

### TECHNICAL FIELD

The present invention relates to a dental laser treatment apparatus, and more particularly to a dental laser treatment apparatus that enables treatment for a tooth or the interior of a mouth, sterilization and cleaning within a mouth, and desensitization to be deftly performed.

### BACKGROUND

A dental laser treatment apparatus using laser beams is widely known. The dental laser treatment apparatus is used for cleaning within a mouth, for example, to remove calculus and plaque within the mouth. The dental laser treatment apparatus is also used for incision of gums, desensitization procedures, and the like in order to treat, for example, periodontal disease and caries. In dental treatment with laser beams, the dental laser treatment apparatus has many advantages, which are features of the dental laser treatment apparatus. The advantages are, for example, that treatment can be performed without injection of the anesthetic as before, no pain is caused by a surgical tool such as a scalpel, bleeding amount is less than that in a common incision treatment, and treatment time can be shortened.

A laser treatment apparatus used for dental treatment includes, for example, an Erbium YAG laser, which is used for treatment of hard tissues such as teeth. In addition, the Erbium laser is used for removing calculus and bacteria accumulated in a periodontal pocket in treatment of periodontal disease. In the case, irradiation is performed by everting gums at the entrance of the periodontal pocket. The Erbium laser is suitable for a case where treatment is performed by directly inserting an instrument to the periodontal pocket, and applying laser beams.

For example, a neodymium YAG laser (Nd: YAG laser) is also used for treatment of soft tissues such as gums. The laser is used for treatment by instantly evaporating pus accumulated in gums due to advanced periodontal disease. Some such techniques for treating teeth with laser beams have been proposed. For example, JP 2010-68880 A (Patent Literature 1) discloses a dental laser treatment apparatus including: an apparatus body including a light source unit that emits a laser beam; an optical fiber that guides the laser beam from the light source unit to a part to be treated; a discharging unit that discharges suspension of metal or particles for metal oxide substances (titanium oxide) at a distal portion of the optical fiber; a suspension supply unit that supplies suspension to the discharging unit. JP 2004-242841 A (Patent Literature 2) also discloses a dental laser treatment apparatus that can efficiently remove plaque.

By the way, in a dental treatment, a patient is required to receive the treatment with his/her mouth kept opened for a certain time. The time varies depending on the skill of a doctor in charge or the difficulty of the treatment. Although the laser treatment apparatuses as described above greatly shorten the time taken for treatment, opening a mouth for a long time puts a burden on the patient. One of the causes is that operations in, for example, a treatment of periodontal disease has been separately performed before. The operations include removal of bacteria crammed in a periodontal pocket and accumulated blood by incising gums, subsequent cleaning of the periodontal pocket, and disinfection of the interior of a mouth including gums. The separately performed operations tend to result in longer operation time.

Introduction of the dental laser treatment apparatuses enable incision and disinfection of gums to be simultaneously performed during a laser treatment, and shortened treatment time reduces burdens on a patient.

Unfortunately, in order to efficiently perform a treatment with laser beams, a dirty part of, for example, protein covering the surface of a part to be irradiated, that is, a part to be treated is required to be removed, and another device has been needed for cleaning the interior of a mouth before. In addition, a wound caused by the laser beams is immediately closed after a laser treatment for gums. In cleaning, only the surface of the gums and a tooth within a mouth are thus cleaned. Cleaning of the back of a periodontal pocket or the interior of gums is difficult. Furthermore, healing after the treatment is slow.

### SUMMARY

The invention is made in view of the above-described problems, and an object thereof is to provide a dental laser treatment apparatus that can facilitate sterilization and cleaning work in a treatment with laser beams and also during and after the treatment, puts less physical and mental burdens on a patient, offers a prospect of quick and reliable therapeutic effects, and allows for faster healing.

The invention made for solving the above-described problems is a dental laser treatment apparatus used during a treatment of, for example, periodontal disease with a laser beam, including: an apparatus body including a laser beam generating device that generates a laser beam; a laser beam application device including an optical fiber that supplies the laser beam generated by the laser beam generating device and a handpiece that includes an application port for the laser beam attached on a distal portion of the optical fiber; and a cleaning solution supply unit that discharges sterilizing/cleaning solution from a sterilizing/cleaning solution supply unit, the sterilizing/cleaning solution supply unit being disposed in the apparatus body or separately disposed, the apparatus body including a supply pipe equipped with a handpiece at a distal end, in which the sterilizing/cleaning solution is aqueous solution containing hypochlorous acid and a hypochlorous acid ion.

According to the invention, sterilizing/cleaning solution having sterilizing action has sterilizing action, and a cleaning solution supply unit is provided in an apparatus body. Sterilizing/cleaning work can thus be easily performed in a treatment with laser beams and also during and after the treatment.

Furthermore, in the invention, in the case where the sterilizing/cleaning solution is aqueous solution whose effective residual chlorine concentration is adjusted to 500 to 2000 ppm, whose hydrogen ion concentration index is adjusted to pH 8.5 to pH 9.5, and whose temperature is adjusted to 40 to 50° C., dirty deposit of, for example, protein covering an affected part can be decomposed and quickly removed before the treatment, so that treatment with laser beams can be highly efficiently and reliably performed. In addition, pain is relieved by applying the sterilizing/cleaning solution to the operation part as necessary during the treatment with laser beams, and thus physical and mental burdens on a patient is eliminated. Furthermore, hemostatic effect is increased, and healing of a wound is fast.

Furthermore, in the case where a storing part for suspension of metal or metal oxide substance particles disposed in an apparatus body or separately disposed is provided, and a suspension supply unit including a handpiece for suspension for supplying the suspension at a distal end is provided, application of laser beams accompanying use of the suspension causes desensitization (dulls sense/perception at an operation part), and no pain is felt.

In addition, in the case where at least one of a handpiece for sterilizing/cleaning solution and a handpiece for suspension is integrated with the handpiece for laser beams, application of laser beams, supply of sterilizing/cleaning solution, and supply of suspension can be operated simultaneously or separately in sequence, with one handpiece kept gripped with the handpiece kept gripped, whereby a highly efficient treatment with laser beams can be performed.

According to a dental treatment apparatus using laser beams of the invention, sterilization and cleaning work can be easily performed in a treatment with laser beams and also during and after the treatment, less physical and mental burdens are put on a patient, a prospect of quick and reliable therapeutic effects is offered, and healing is fast.

### BRIEF DESCRIPTION OF THE DRAWINGS

FIG. 1 is a schematic view illustrating a preferred embodiment of the invention.

FIG. 2 is an enlarged partial view of a nozzle disposed at the distal end of a handpiece in the embodiment illustrated in FIG. 1.

FIG. 3 illustrates a surface of the distal end in FIG. 2.

FIG. 4 illustrates a surface of the distal end of another handpiece in the embodiment illustrated in FIG. 1.

FIG. 5 is a schematic view illustrating another embodiment of the invention.

FIG. 6 is an enlarged partial view of a nozzle disposed at the distal end of a handpiece in the embodiment illustrated in FIG. 5.

The invention will now be described in detail based on the preferred embodiments illustrated in the drawings.

### DETAILED DESCRIPTION

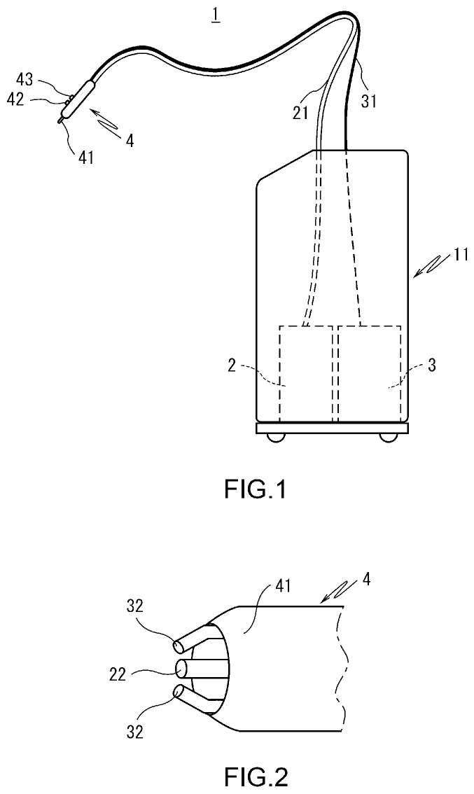

FIG. 1 schematically illustrates a dental laser treatment apparatus 1 with a sterilizing/cleaning function of the invention. A case-shaped apparatus body 11 includes a laser beam generating device 2 and a sterilizing/cleaning solution storing tank 3. The laser beam generating device 2 generates laser beams.

Furthermore, the laser beam generating device 2 includes full specification pulses for a medical laser. The laser beam generating device 2 can apply the generated laser beams from an application nozzle 22 for the laser beams of a composite nozzle 41, which is provided at a distal end of a handpiece 4, via an optical fiber 21 while selecting laser beams of various wavelengths necessary for a treatment with, for example, an adjustment device (not illustrated) provided in the apparatus body 11.

In addition, the sterilizing/cleaning solution storing tank 3 is connected to a sterilizing/cleaning solution discharging port 32 via a supply pipe 31. The sterilizing/cleaning solution discharging port 32 is disposed alongside the application nozzle 22 for the laser beams of the composite nozzle 41, which is provided at the distal end of the handpiece 4. The sterilizing/cleaning solution is supplied from the sterilizing/cleaning solution discharging port 32 to an affected part within a mouth through a delivery pump (not illustrated).

Note that, although, in the embodiment, the sterilizing/cleaning solution storing tank 3 is disposed in the apparatus body 11 as a cleaning solution supply unit, a sterilizing/cleaning solution supply unit may be disposed separately from the apparatus body 11 (not illustrated).

In addition, in terms of sterilizing power, the sterilizing/cleaning solution is preferably aqueous solution electrolytically generated from an electrolyte made from, for example, ultrapure water and sodium chloride added in the ultrapure water. The effective residual chlorine concentration of the aqueous solution is adjusted to 500 to 2000 ppm, the hydrogen ion concentration index thereof is adjusted to pH 8.5 to pH 9.5, and the temperature thereof is adjusted to 40 to 50° C.

Ultrapure water having an electric resistivity of 15 MΩ cm or more at a water temperature of 25° C. and a total organic carbon (TOC) of less than 0.05 mg/L is used for the sterilizing/cleaning solution of the invention. This causes migration of ions and decomposition of water (H₂O). Oxygen (O₂) gas is generated and the concentration of hydrogen (H) ions is increased on the positive-electrode side. Hydrogen (H₂) gas is generated and the concentration of hydroxide (OH) ions is increased on the negative-electrode side. In the case, however, electrolysis is performed with no diaphragm, so that chlorine generated on the positive-electrode side reacts with high-concentration sodium hydroxide generated on the negative-electrode side to generate sodium hypochlorite.

The process is expressed in the following expression.

Negative-electrode side: 2Na₂+2H₂O+2e→2NaOH+H₂

Positive-electrode side: 2Cl→Cl₂+2e

As a result, the reaction between chlorine and sodium hydroxide occurs, and the following expression is considered to be satisfied.

2NaOH+Cl₂→NaClO+NaCl+H₂O

A state of a biofilm on the inside surface of a tube included in a dental unit before testing was compared to the condition of the biofilm inside the tube two weeks after circulation of sterilizing/cleaning solution. Specifically, three cases were compared. One was a case that was defined as a control. Another was a case where a tube was cleaned with sterilizing/cleaning solution. The other was a case where sterilizing/cleaning solution is used in a fluorine-coated tube.

Shown is a photograph illustrating a state of bacteria attached on the inside surface of the tube after the sterilizing/cleaning solution is used for cleaning the interior of the tube of the dental medical instrument. In the control, two weeks had elapsed since the start of the testing, and the bacteria had further grown to form a biofilm. In contrast, in the case where the sterilizing/cleaning solution according to the embodiment was used for cleaning the interior of the tube, the biofilm of bacteria like that seen at the start of the testing had not been formed at the time when two weeks had elapsed.

Note that the sterilizing/cleaning solution for the invention having a water temperature of 40 to 50° C. (preferably about 45° C.) does not irritate an affected part, and can have a great sterilizing/cleaning effect. Preferably, for example, the sterilizing/cleaning solution storing tank 3 is warmed, or an electronic instantaneous heating device (not illustrated) is attached on the supply pipe 31 in usage.

In particular, in the embodiment, electrical switches 42 and 43 for controlling the application of laser beams and supply of sterilizing/cleaning solution are provided side by side on the handpiece 4. The application of laser beams or the supply of sterilizing/cleaning solution can be controlled simultaneously or separately with the handpiece 4 kept gripped.

Consequently, in the present embodiment, an affected part within a mouth or the affected part after a treatment can be treated quickly and cleanly while being sufficiently sterilized by the sterilizing/cleaning solution before or simultaneously with the treatment using laser beams.

In particular, in the case where the cleaning solution is aqueous solution whose effective residual chlorine concentration is adjusted to 500 to 2000 ppm, whose hydrogen ion concentration index is adjusted to pH 8.5 to pH 9.5, and whose temperature is adjusted to 40 to 50° C., dirty deposit of, for example, protein covering the affected part can be decomposed and quickly removed before the treatment. Treatments such as incision or burn by using laser beams directly applied to the affected part can be performed with the affected part exposed by pouring the cleaning solution from a cleaning solution discharging port to the affected part at the start of the treatment. An efficient, non-wasteful, and reliable treatment can be performed.

In addition, the sterilizing/cleaning solution supplied during the treatment with laser beams exhibits cooling or buffering effect, so that a patient feels no pain even at the time when tissues within the mouth are incised or excised in the treatment. The patient has no physical and mental suffering. Furthermore, burned tissues of the affected part and dirt generated in the treatment is peeled off or eluted, and the laser beams can be directly applied to the affected part.

Of course, a treatment-caused scar is sufficiently sterilized and kept clean, and no film of dirty deposit is formed on the surface due to the sterilizing/cleaning solution supplied over, for example, the burned scar after the treatment. As a result, healing is fast, and there is no need to worry about suppuration of the affected part after the treatment.

Note that the invention that uses the sterilizing/cleaning solution is far superior to a comparative example that uses traditional cleaning solution such as water. For example, in a treatment for removing a part of a male adult affected by periodontal disease bacteria, a specific actual example will be shown in the following table in comparison with a comparative example performed by using cleaning solution such as common tap water through another cleaning nozzle.

TABLE 1

| | | Comparative |
|---|---|---|
| | Present invention | example |
| Cleaning solution | Sterilizing/Cleaning | Tap water |
| | solution | |
| Treatment time | Approximately | Approximately |
| | 20 minutes | 30 minutes |
| Pain during treatment | Little | A little |
| Hemostatic condition | Bleeding stops at the same | Bleeding continues |
| | time as treatment is | for several weeks |
| | finished | |
| Healing condition of | Approximately several | Approximately |
| wound | days | one week |

Table 1 indicates that the invention is far superior to the traditional comparative example in therapeutic effects.

Note that, in the case where the sterilizing/cleaning solution in the invention is aqueous solution whose effective residual chlorine concentration is adjusted to 500 to 2000 ppm, whose hydrogen ion concentration index is adjusted to pH 8.5 to pH 9.5, and whose temperature is adjusted to 40 to 50° C. as described above, more excellent action and effect can be exhibited. Sterilization and cleaning to an incised part with sterilizing/cleaning solution performed at substantially the same time as incision with laser beams for gums swollen by periodontal disease enables cleaning of the interior of the gums that has not performed before. A treatment time for periodontal disease can be greatly shortened, and burdens on a patient can be reduced. Traditional cleaning with tap water does not exhibit such effects. In particular, the sterilizing/cleaning solution discharging port 32 enables sterilization and cleaning performed at the same time as a treatment with laser beams. The sterilizing/cleaning solution discharging port 32 is disposed alongside the application nozzle 22 for laser beams of the composite nozzle 41, which is provided at the distal end of the one handpiece 4. A disinfection effect within a mouth and gums is further enhanced.

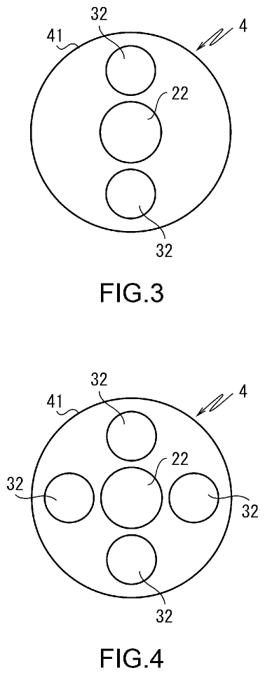

Note that, in the embodiment, one application nozzle 22 for laser beams and a plurality of (e.g., two in FIG. 3, and four in the embodiment in FIG. 4 sterilizing/cleaning solution discharging ports 32 around the application nozzle 22 are disposed in one handpiece 4, so that a desired appropriately selected amount can be used at a desired position and a desired discharge width by appropriately opening and closing these sterilizing/cleaning solution discharging ports 32 with an operation switch (not illustrated).

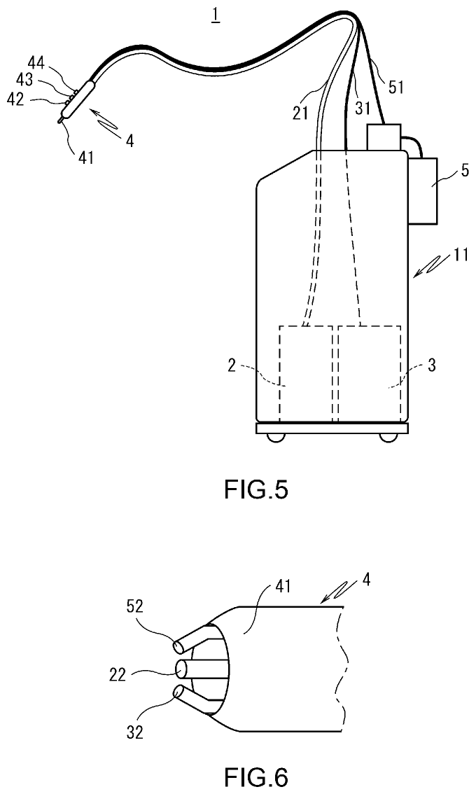

FIG. 5 schematically illustrates a dental laser treatment apparatus 1 with a sterilizing/cleaning function in another embodiment of the invention. A case-shaped apparatus body 11 includes a laser beam generating device 2, a sterilizing/cleaning solution storing tank 3, and a suspension storing tank 5. The laser beam generating device 2 generates laser beams. The suspension contains, for example, metal or metal oxide substance particles such as titanium and titanium oxide.

Furthermore, the laser beam generating device 2 includes full specification pulses for a medical laser. The laser beam generating device 2 can apply the generated laser beams from an application nozzle 22 for the laser beams of a composite nozzle 41, which is provided at a distal end of a handpiece 4, via an optical fiber 21 while selecting laser beams of various wavelengths necessary for a treatment with, for example, an adjustment device (not illustrated) provided in the apparatus body 11. In particular, the laser beam generating device 2 can process the applied laser beams such that the applied laser beams have a top shape suitable for a treatment type or part, for example, a substantially conical shape and a substantially truncated conical shape, and use the laser beams while supplying suspension from the suspension storing tank 5 via a supply pipe 51 from a suspension discharging port 52. The suspension discharging port 52 is disposed along the application nozzle 22 for laser beams in the composite nozzle 41.

In addition, the sterilizing/cleaning solution storing tank 3 is connected to a sterilizing/cleaning solution discharging port 32 via a supply pipe 31. The sterilizing/cleaning solution discharging port 32 is disposed alongside the application nozzle 22 for the laser beams of the composite nozzle 41, which is provided at the distal end of the handpiece 4. The cleaning solution is supplied from the sterilizing/cleaning solution discharging port 32 to an affected part within a mouth through a delivery pump (not illustrated).

In particular, in the embodiment, electrical switches 42, 43, and 44 for controlling the application of laser beams, supply of sterilizing/cleaning solution, and supply of suspension are provided side by side on the handpiece 4. The application of laser beams, the supply of sterilizing/cleaning solution, and the supply of suspension can be controlled simultaneously or separately with the handpiece 4 kept gripped.

Note that, as in the above-described embodiment illustrated in FIGS. 1 to 4, in terms of sterilizing power, the sterilizing/cleaning solution is preferably aqueous solution electrolytically generated from an electrolyte made from, for example, ultrapure water and sodium chloride added in the ultrapure water. The effective residual chlorine concentration of the aqueous solution is adjusted to 500 to 2000 ppm, the hydrogen ion concentration index thereof is adjusted to pH 8.5 to pH 9.5, and the temperature thereof is adjusted to 40 to 50° C. In addition, usage of ultrapure water having an electric resistivity of 15 MΩ cm or more at a water temperature of 25° C. and a total organic carbon (TOC) of less than 0.05 mg/L causes migration of ions and decomposition of water (H₂O). Oxygen (O₂) gas is generated and the concentration of hydrogen (H) ions is increased on the positive-electrode side. Hydrogen (H₂) gas is generated and the concentration of hydroxide (OH) ions is increased on the negative-electrode side. In the case, however, electrolysis is performed with no diaphragm, so that chlorine generated on the positive-electrode side reacts with high-concentration sodium hydroxide generated on the negative-electrode side to generate sodium hypochlorite. A water temperature of 40 to 50° C. (preferably about 45° C.) does not irritate an affected part, and can have a great sterilizing/cleaning effect. For example, the sterilizing/cleaning solution storing tank 3 is warmed, or an electronic instantaneous heating device (not illustrated) is attached on the supply pipe 31 in usage.

Furthermore, in the embodiment, dirty deposit of, for example, protein covering the affected part can be decomposed and quickly removed before the treatment. Treatments such as incision or burn by using laser beams directly applied to the affected part can be performed with the affected part exposed by pouring the sterilizing/cleaning solution from a cleaning solution discharging port to the affected part at the start of the treatment. An efficient, non-wasteful, and reliable treatment can be performed.

In addition, the sterilizing/cleaning solution supplied during the treatment with laser beams exhibits cooling or buffering effect, so that a patient feels no pain even at the time when tissues within the mouth are incised or excised in the treatment. The patient has no physical and mental suffering. Furthermore, burned tissues of the affected part and dirt generated in the treatment is peeled off or eluted, and the laser beams can be directly applied to the affected part.

In this point, as in the above-described embodiment, a treatment-caused scar is sufficiently sterilized and kept clean, and no film of dirty deposit is formed on the surface due to the sterilizing/cleaning solution supplied over, for example, the burned scar after the treatment. As a result, healing is fast, and there is no need to worry about suppuration of the affected part after the treatment. Sterilization and cleaning to an incised part with sterilizing/cleaning solution performed at substantially the same time as incision with laser beams for gums swollen by periodontal disease enables cleaning of the interior of the gums that has not performed before. A treatment time for periodontal disease can be greatly shortened, and burdens on a patient can be reduced. Traditional cleaning with tap water does not exhibit such effects. In particular, the sterilizing/cleaning solution discharging port 32 enables sterilization and cleaning performed at the same time as treatment with laser beams. The sterilizing/cleaning solution discharging port 32 is disposed alongside the application nozzle 22 for laser beams of the composite nozzle 41, which is provided at the distal end of the one handpiece 4. A disinfection effect within a mouth and gums can be further enhanced.

In addition, in the embodiment, a case-shaped apparatus body 11 includes a laser beam generating device 2, a sterilizing/cleaning solution storing tank 3, and a suspension storing tank 5. The laser beam generating device 2 generates laser beams. The suspension contains, for example, metal or metal oxide substance particles such as titanium and titanium oxide.

Furthermore, the laser beam generating device 2 includes full specification pulses for a medical laser. The laser beam generating device 2 can apply the generated laser beams from an application nozzle 22 for the laser beams of a composite nozzle 41, which is provided at a distal end of a handpiece 4, via an optical fiber 21 while selecting laser beams of various wavelengths necessary for a treatment with, for example, an adjustment device (not illustrated) provided in the apparatus body 11. In particular, the laser beam generating device 2 can process the applied laser beams such that the applied laser beams have a top shape suitable for a treatment type or part, for example, a substantially conical shape and a substantially truncated conical shape, and use the laser beams while supplying suspension from the suspension storing tank 5 via a supply pipe 51 from a suspension discharging port 52. The suspension discharging port 52 is disposed along the application nozzle 22 for laser beams in the composite nozzle 41.

Furthermore, excellent effects of the embodiment can be exhibited in usage for periodontal disease treatment as follows. A treatment example therefor will now be described in detail together with effects.

(Step 1) Preoperative Cleaning

First, the interior of a mouth is sterilized and cleaned by sterilizing/cleaning solution from the sterilizing/cleaning solution discharging port 32 of the composite nozzle 41 as preoperative procedure.

(Step 2) Desensitization

An affected part is desensitized by using laser beams and suspension together. The laser beams are applied from the application nozzle 22 for the laser beams of the composite nozzle 41, which is provided at a distal end of the handpiece 4. The suspension contains metal or metal oxide such as titanium and titanium oxide, and is discharged from the suspension discharging nozzle 52. The suspension discharging nozzle 52 is disposed along the application nozzle 22 for laser beams in the composite nozzle 41, which is disposed at the distal end of the handpiece 4.

(Step 3) Calculus Removal and Sterilization of Interior of Mouth

The interior of a periodontal pocket is cleaned, and calculus on the edge of gums is removed with an ultrasonic scaler while sterilizing/cleaning solution is spouted from the sterilizing/cleaning solution discharging port 32 disposed at the distal end of the handpiece 4. The effective residual chlorine concentration of the sterilizing/cleaning solution is adjusted to 500 to 2000 ppm, the hydrogen ion concentration index thereof is adjusted to pH 8.5 to pH 9.5, and the temperature thereof is adjusted to 40 to 50° C. At the time, the interior of the mouth is instantaneously sterilized simultaneously with the calculus removal.

(Step 4) Removal of Calculus on Tooth Root Surface

Furthermore, thorough root planning is performed with a tool for removing calculus on a tooth root surface. The above-described desensitization condition is maintained at the time, and no pain is felt.

(Step 5)

Stubborn stains, biofilms, and colored stains sticking between teeth are swept away by vigorously spraying superfine powder on a tooth surface with a separately equipped handy jet.

(Step 6) Mechanical Tooth Surface Cleaning and Sterilization

The tooth surface is smoothed by mechanical tooth surface cleaning so as to be plaque-resistant, and at the same time, enamel is strengthened. After the completion, the interior of the periodontal pocket is cleaned and sterilized again.

(Step 7) Fluorine Coating

The cleaned tooth surface is coated with fluorine by a tray technique.

(Step 8) Laser Beam Application

Laser beams are applied from the application nozzle 22 for the laser beams of the composite nozzle 41, which is provided at a distal end of the handpiece 4 so that a blood flow within the gums and the mouth is activated. As a result, healing of periodontal tissues is accelerated, weakened immunity is restored, pain is removed, and recovery from exhaustion is obtained. Note that, in the embodiment, the suspension discharging port 52 is disposed along the application nozzle 22 for the laser beams of the composite nozzle 41, which is provided at a distal end of the handpiece 4, as the invention disclosed in JP 2010-68880 A. Laser beams are switched, and desired laser beams can be applied while suspension is used.

Although one example of a treatment with a dental laser treatment apparatus of the invention has here been described above, the dental laser treatment apparatus of the invention can be used for various treatments such as dental treatments and mouth interior treatments. In particular, the dental laser treatment apparatus is equipped with sterilizing/cleaning solution, and enables constant sterilization and cleaning. In particular, the dental laser treatment apparatus can have massive action and effect compared to a case where traditional tap water, which is relatively weak and easily polluted, is used.

1. A dental laser treatment apparatus used during a treatment of periodontal disease with a laser beam, comprising:
an apparatus body including a laser beam generating device that generates the laser beam;
a laser beam application device including an optical fiber that supplies the laser beam generated by the laser beam generating device and a first handpiece that includes an application port for the laser beam attached on a distal portion of the optical fiber; and
a cleaning solution supply unit that discharges a solution for at least one of sterilizing and cleaning, which is aqueous solution containing hypochlorous acid and a hypochlorous acid ion, from a solution supply unit, the solution supply unit being disposed in the apparatus body or separately disposed, the apparatus body including a supply pipe equipped with a second handpiece at a distal end,
wherein the second handpiece for the solution is integrated with the first handpiece for the laser beam, a treatment with the laser beam is allowed to be performed at a same time as the at least one of sterilization and cleaning, four solution discharging ports are disposed in longitudinal and lateral directions about an application nozzle for the laser beam, and a desired amount is allowed to be discharged by an operation switch from a desired solution discharging port at a desired discharge width.

2. The dental laser treatment apparatus according to claim 1, wherein the solution is an aqueous solution whose effective residual chlorine concentration is adjusted to 500 to 2000 ppm, whose hydrogen ion concentration index is adjusted to pH 8.5 to pH 9.5, and whose temperature is adjusted to 40 to 50° C.

3. The dental laser treatment apparatus according to claim 1, further comprising:
a storing part for suspension of metal or metal oxide substance particles disposed in the apparatus body or separately disposed; and
a suspension supply unit including a third handpiece for suspension that supplies the suspension at a distal end.

4. The dental laser treatment apparatus according to claim 1, wherein the second handpiece for the at least one of sterilizing and cleaning the solution and a third handpiece for suspension are integrated with the first handpiece for the laser beam.

5. The dental laser treatment apparatus according to claim 2, further comprising:
a storing part for suspension of metal or metal oxide substance particles disposed in the apparatus body or separately disposed; and
a suspension supply unit including a third handpiece for suspension that supplies the suspension at a distal end.

6. The dental laser treatment apparatus according to claim 2, wherein the second handpiece for the at least one of sterilizing and cleaning the solution and a third handpiece for suspension are integrated with the first handpiece for the laser beam.

7. The dental laser treatment apparatus according to claim 3, wherein the second handpiece for the at least one of sterilizing and cleaning the solution and the third handpiece for suspension are integrated with the first handpiece for a laser beam.
